(19) United States
(12) Patent Application Publication
(10) Pub. No.: US 2021/0074389 A1
(43) Pub. Date: Mar. 11, 2021

(54) SYSTEM AND METHOD FOR COLLECTING, ANALYZING, AND UTILIZING COGNITIVE, BEHAVIORAL, NEUROPSYCHOLOGICAL, AND BIOMETRIC DATA FROM A USER'S INTERACTION WITH A SMART DEVICE WITH EITHER PHYSICALLY INVASIVE OR PHYSICALLY NON-INVASIVE MEANS

(71) Applicant: Shoestring Psychometrics, North Las Vegas, NV (US)

(72) Inventors: Tyler Raymond Parker, North Las Vegas, NV (US); Rene Antonio Diaz, Humble, TX (US)

(73) Assignee: Shoestring Psychometrics, North Las Vegas, NV (US)

(21) Appl. No.: 17/019,116

(22) Filed: Sep. 11, 2020

Related U.S. Application Data
(60) Provisional application No. 62/898,770, filed on Sep. 11, 2019.

(51) Int. Cl. G16H 10/20; G16H 40/67; A61B 5/04

(52) U.S. Cl. CPC G16H 10/20; A61B 5/04001; G16H 40/67

(57) ABSTRACT

A software utility that collects a suite of psychobehavioral, neuropsychological, and biometrically relevant data from neuropsychological tests, and from passive and active interaction with a smart device. Passive interaction is a user's interaction that is not explicitly goal directed. Active interaction is explicitly goal directed (e.g., navigating menus, or interacting with an application). This data is used to: 1) provide an objective profile of memory, cognition, perception, motor function, verbal ability, and fluid intelligence; 2) adapt hardware, software, and user interface settings to make informed decisions regarding accessibility options; 3) to detect usage by someone other than the native user of the device, and 4) to provide a unifying protocol (e.g. an API) for the transmission and receipt of data collected from onboard sensor arrays and software—for processing either locally or remotely.

### CROSS-REFERENCE TO RELATED APPLICATIONS

This application claims priority under 35 U.S.C. § 119(e) from co-pending U.S. Provisional Patent Application No. 62/898,700, by Parker & Diaz, “System and Method for Collecting, Analyzing, and Utilizing Neuropsychological and Biometric Data from a User's Interaction with a Smart Device” filed 11 Sep. 2019, which by this statement, is incorporated herein by reference for all purposes.

### STATEMENT REGARDING FEDERALLY SPONSORED RESEARCH OR DEVELOPMENT

Not Applicable.

### REFERENCE TO SEQUENCE LISTING, A TABLE, OR A COMPUTER PROGRAM LISTING COMPACT DISC APPENDIX

Not Applicable.

### BACKGROUND OF THE INVENTION

### Field of the Invention

The present innovation relates to the field of acquiring, analyzing, modifying, and utilizing cognitive, behavioral, neuropsychological, and biometric data relevant to functioning of an individual and/or group. More specifically, the innovation concerns the use of smart-enabled devices containing a variety of sensor arrays to accomplish the relevant data, details and examples of which are described below.

### Background of the Invention

In the field of neuropsychological test administration and assessment, specifically with commonly used paper-and-pencil test administrations and assessments the processes of data collection, analysis, and utilization can be a cumbersome and painstaking endeavor. Neuropsychologists use neuropsychological testing to assess an array of functions and characteristics of human behavior and cognition including, but not limited to: memory, cognition, perception, motor function, verbal ability, fluid intelligence, and others.

Computerized neuropsychological test batteries exist and are in use today, but they are specific applications which test subjects interact with for the purpose of gathering the data. This environment means the test subjects are clearly aware of the testing activity, and thus may skew the test data either consciously or subconsciously through their actions. For example, an AMID subject may use intense focus on the task, as a way to compensate for or deter a normal activity of “day-dreaming”.

A neuropsychologist, or any other skilled or unskilled test administrator must monitor a number of particular elements of testing simultaneously including, but not limited to: the fidelity of the test administration to the testing protocol; a test participant's continual progress during the test; timing (preferably with millisecond precision); perceived level of participant's difficulty and/or frustration while undertaking the task; errors made and corrected; errors made and not corrected; omissions and deviations from the testing process; methods to adapt the test to a participant's accessibility requirements; any and all relevant clinical observations; adaptation to any unanticipated irregularities to the testing environment; and many other factors not explicitly mentioned in this list.

This is typically all done by a single neuropsychologist, skilled test administrator, or unskilled test administrator. Data traditionally collected and analyzed by a neuropsychologist during a single test administration includes but is not limited to: score-based performance on a test; time taken to complete a test; clinical and/or behavioral observations noted during a test; as well as other factors not explicitly listed here.

The data collected within a test (or within a series of tests, referred to in the art as a “test battery”) is then used to make clinical determinations regarding a the test subject's performance measures including, but not limited to: memory, cognition, perception, motor function, verbal ability, fluid intelligence, and others.

Another difficulty with traditional neuropsychological assessments is the degree of test sensitivity to detecting minuscule, but significant, differences in participant performance which can contribute to differential outcomes for individuals with similar score profiles. This is enhanced by the manual testing and observation methods, particularly by unskilled test administrator.

In the field of user interface design and adaptation via accessibility features, user interfaces on smart devices will only change their characteristic design and presentation after a user selects these adaptations between a variety of these options—typically by visual presentation on a screen, but sometimes through, audio. Smart devices do not currently automatically adapt to a user's cognitive, behavioral, neuropsychological or biometric response patterns and input.

As an example, if a person with vision loss needs to configure audio responses, they must get assistance to set the correct accessibility settings since doing so requires a user navigate through the accessibility options via a menu presented visually on the screen. Alternatively a person who loses a sufficient amount of their previous cognitive functioning (for example due to a concussion) may find it difficult, or even impossible, to interact with their device in their usual manner.

This person is essentially restarting the process of learning to use their device. Such a task would be much easier and faster if the device were already aware of the person's prior usage patterns and could use those patterns along with current prediction software capabilities to accurately predict intentions or activity to more quickly be able to provide the desired responses.

Smart devices are not, currently, sensitive to or adaptable enough to these acute (or even gradual) changes in an individual's neuropsychological and/or behavioral profiles over time. This means they are not able to automatically account for these changes and adapt accessibility settings accordingly. Users are not typically made aware by the device that their behavior when interacting with the device has undergone a detectable change.

In the field of smart device security, smart devices are nut capable of discriminating between users unless they specifically identify themselves. By using cognitive, behavioral, neuropsychological and biometric differences, a device can discriminate between users, even when the user applies deception techniques. This security may be applied in active security features such as requiring a secondary verification to ensure authorized access by the native user.

This, technology can also be used to apply minor modifications where multiple users are anticipated. For example, when a child unlocks their parent's smart device, the child has full access to the device. Parents and their children interact differently with the same smart device, but the smart device was previously unable to detect the differences. After determining the user is not the native user (the parent) it can apply certain security measures such as not allowing execution of financial apps or limiting content rating on entertainment apps.

Alternatively, if an individual that is not the native user of the smart device gains access to the smart device, the smart device is not able to discriminate this non-native user from the native user based on input and feedback sequences inherent in their interaction with the device. This technology could detect a change in an active user's measurements, and upon determination that a user change has occurred, the device could perform a lock out and require reverification.

In the field of academic assessment, the only relevant test inputs are time taken to complete an assessment and the percentage of correct to incorrect responses. There is no currently existing method to track, via visual gaze heat mapping, how and where a test-taker visually scans (or skips) information presented on an assessment. This information, if provided, can help educators to identify cognitive processes undertaken by a test-taker as they complete an assessment.

In the field of video gaming and e-sports, there is not an integrated method within gaming applications to measure cognitive, behavioral, and neuropsychological functions such as visual scanning patterns; ratio of seen targets to unseen targets; latency between visual detection of a target and a “click” or other interaction; efficiency of movement and motion within the in-game environment and the behavior of the eyes; etc. There is minimal eye gaze tracking technology within some gaming applications, but they are inferior to those presented here.

In the field of behavioral analytics, a user's preferences come from their interaction with data elements presented to them via “liking” a page or visiting a website. In the present state of the art, user gaze tracking or orienting the display device towards or away from the user's gaze is not part of the repertoire of determining a user's preferences while viewing content.

### BRIEF SUMMARY OF THE INVENTION

A problem stated above is that smart devices are incorporating larger sensor arrays, but the data these sensor arrays generates is not accessible to the general applications, and specialty applications are limited to the data of a few specific sensors. There is limited ability to collect biometric data upon request by a software application, and then only from the specific sensors the application is configured to access.

By making biometric data an integral part of smart enabled device functionality, there exists the ability to analyze and utilize biometric data within the fields of neuropsychological assessment, smart device accessibility settings, smart device security, academic assessment, video gaming, behavior analytics, and other implementations not explicitly mentioned here. An important aspect is psychometric assessment to directly correlate user behavior and neuropsychological state. Such tracked behavior can be directly attributed to specific brain states and functions over time.

The innovation presented here is a unifying software system and method that collects an array of cognitive, behavioral, neuropsychological, and biometrically relevant data from an array of user interaction modalities, including, but not limited to: neuropsychological assessments; smart device accessibility settings; smart device security; academic assessments; video gaming; behavior analytics; web browsing; general smart device application usage; and other related activities. This innovation allows linking a user's overt behavior with a device to the functioning of that or another device and the resulting data streams.

Herein the term “smart device” describes an electronic computing device having a micro-controller or a computer that is configurable or programmable to perform different task or run different applications. These devices are generally interconnected or networked with other similar devices to communicate and share data in real time, but this is not a necessity for some aspects of the innovation described herein. A smart device may be referenced as a “device” for simplicity but has the same meaning unless the context of the usage designates otherwise.

Through an Application Programming Interface (an “API”) the innovation makes accessible data from a device's sensor array and presents it for diverse uses. The data collected comprises an array of cognitive, behavioral, neuropsychological, and biometrically relevant data from an array of user interaction modalities including, but not limited to: neuropsychological assessments; smart device accessibility settings; smart device security; academic assessments; video gaming; behavior analytics; web browsing; general smart device application usage; and other related activities.

Passive and active user data collection; User behavioral data is collected through a device's sensor arrays by passive and active means. Passive interaction is defined as interaction that is not explicitly goal directed. Examples include: placing the device in the user's pocket; setting the device on a table, turning the device's screen away from the face (i.e. not tending to it); physically transporting the device (carrying it from location to location). Active interaction is interaction that is explicitly goal directed. Examples include: navigating through menus, pressing buttons, using a software application, viewing the device's screen (particularly shifting eye gaze across the screen display).

Passively and actively collected cognitive, behavioral, neuropsychological, and biometric data provides an objective neurocognitive profile of memory, cognition, perception, motor function, verbal ability, fluid intelligence, habit, and other measurements. This profile may be used for advertising; security; video gaming; automatically adapting user interface settings based on assessed needs; and other uses.

Examples of user input data collected from device interfaces includes, but is not limited to: tactile (touch, click, or input); duration of tactile input; tactile force input profiles over time intervals; number of inputs over time intervals; route and direction of tactile input; specific segments of fingerprints used to touch a screen (to determine the angle of incidence of specific fingers with the screen); infrared and/or “heat” map data taken from interaction with the screen (tactile and eye gaze tracking)

Examples of device data related to a user of the device includes, but is not limited to: gyroscopic data; accelerometer data; ultrasonic positioning system information, such as eye gaze detection, eye disengage detection, eye saccade detection, ocular detections of inattention and/or drowsiness, facial data.

Examples of other data collectable by a device includes but is not limited to: infrared data (detected by a camera); proximity detection; microphone input; GPS data; clock data; specialized sensor inputs such as electrical sensors or electrodes placed and configured to record: the brain producing an Electroencephalogram (aka an “EEG”), the heart producing an Electrocardiogram (aka an “ECG” or “EKG”), or muscles and motor neurons producing an Electromyography (aka an “EMG”), or other sensors for deduction of behavioral, neuropsychological, and biometric data.

Neuropsychological testing: Neuropsychological testing objectives are better realized by non-biased testing precisely measured electronically removing data taint of test administrators, particularly those of unskilled test administrators. Electronic administration can also benefit administering of traditional neuropsychological test or assessment methods.

Data collected over and above that of traditional testing includes: changed responses, time to complete individual task within test, and other revealing sub-activities previously missed. This information can be compiled with the psychobehavioral and biometric data, outlined above, to form a robust set of data from which to perform an analysis. Electronic administration would reduce the time taken to administer neuropsychological tests, reduce errors relating to differences in test administration and scoring.

Data collected from electronic administration would increase the robustness of datasets relevant to behavioral observations collected during neuropsychological testing, increasing the accuracy of recording neuropsychological test results and related biometric measurements, increase the scope and accuracy of the clinical picture and impression of a test participant, naturalistic setting test administration (more closely emulating neuropsychological functioning within a participant's typical environment), allow for a neuropsychologist, skilled, or unskilled test administrator to focus their attention more closely on recording the clinical presentation of the test participant rather than splitting that attention with scoring and tracking the test in real time, as well as other benefits not explicitly listed here.

User interface and accessibility: User interface modification would be accomplished, in an embodiment of the invention, by the active and passive collection of behavioral, cognitive, neuropsychological, and biometrically relevant data retrieved from a user's interaction with a smart device over time in order to make automatic and/or user initiated changes to a smart device's accessibility settings. The accomplishment of this objective would reduce the burden of an impaired person successfully navigating to and selecting appropriate accessibility settings.

Device security Device security and user identification would be accomplished, in an embodiment of the invention, by the collection of behavioral, cognitive, neuropsychological and biometric data by both a user's active and passive interaction with the smart device over a period of time (Time>n). This dataset would then be compared with both active and passive interaction with the smart device over the preceding time period (Time<n) to determine whether the user interacting with the device is the user native to the smart device or some other user. The accomplishment of this objective would lead to increased privacy and security for smart devices, as well as in creating a more robust and accurate dataset relevant to identifying individual smart device users.

Advertising and created content: Advertising and content creator objectives in an embodiment of the invention include using eye gaze beat maps, makers of attention and inattention, GPS data, clock data, EEG, ECG/EKG, EMG and other such sensor data to determine how a user is or isn't attending to specific elements of presented content (including when and where a user attends to these advertisements). This information is used to further capture a user's consumer preferences, unstated likes and dislikes, future needs, and general patterns of consumer behavior.

Video gaming: Video gaming applications in an embodiment of the invention include using eye gaze tracking, cognitive and behavioral observation and analysis, game specific data collection, timing applications, as well as others to assess any number of features of a user's in-game performance using the above stated methods of cognitive, behavioral, neuropsychological, and biometric data collection (as well as closely related other methods).

Academic Assessment: Academic assessment objectives can be accomplished by incorporating eye gaze tracking technology in order to analyze how a user is perceiving and attending to stimulus presented with an audio, visual, or other display. By tracking a user's gaze across stimuli, an educator is able to more closely determine the underlying cognitive process a user is using as they encounter a problem and formulate a solution. This information can be used to design and implement an individualized academic intervention or enhancement plan.

Application Programming Interface: The API is an unifying protocol for the transmission and receipt of data collected from sensor arrays and software data for the purposes of transmitting and receiving, psychobehavioral, neuropsychological, and biometric data sequences processed either locally or remotely. Data collected may be packaged raw, or may be locally analyzed and/or combined, with other data, and the resulting observations made available locally, or packaged alone, or with raw data for storage or transmission for later and/or remote processing.

Objective User Neuropsychological Profile: An objective neuropsychological profile can be derived from a user's interaction with a smart device. A system level application (referred to hereinafter as a “sys-app”) can access, measure, and record user interactions with other apps executing on the device (referred to hereinafter as a “sub-app”). The sys-app can also utilize other sensors that the sub-app does not for additional data collection which can be time synchronized with sub-app data.

The sys-app can collect interaction data by inserting a shim between the device and the application at the system level to record activity before it is reported to the sub-app. This method allows monitoring and analysis of interactions with sub-apps without interfering with performance of the sub-apps and thus possibly skewing a user's behavior.

Layer 1: Stimulus Presentation: The first step in the process of analyzing brain function and creating an objective neuropsychological profile of a user with a smart device is to present some stimulus to the user. A sys-app is one embodiment which allows stimulus to be presented by any sub-app, or by a specifically tailored applications such as those which electronically implement traditional psychological tests.

For example: an image file represented on the device screen may be considered a stimulus presentation. A user's viewing of the image is tracked by the sensors on-board the device. In a traditional psychological test, an image is changed at random time intervals, and the test subject indicates perception of the change, for instance, by pushing a button. Changes in response times can indictive of concentration or focus on the task. In an alternative, a sys-app can use the device's camera to track user eye-gaze to distinguish be concentration or focus issues and muscle control issues preventing quick responses.

In another embodiment, one absent a stimulus, an event or time marker will demarcate the “stimulus” against which a user's behavior will be gauged. For example, if the device sensors detect an abrupt collision with a solid surface, that collision detection can be considered the “stimulus” and sensors can collect input to determine the state of the user. In such an embodiment, as described in the previous paragraph, the event or time marker (an “initiating event”) designates the start of data collection from the sensor array.

Layer 2: Sensor arrays and raw data streams: After the initial stimulus presentation, or the initiating event, the second step in the process is to activate or begin capturing raw data from one or more sensors. Alternatively, the sys-app may be configured to continuously monitor a sensor and maintain a log which could be accessed to analyze a user's behavior occurring prior to stimulus presentation or initiating event.

Such sensors may include, but are not limited to: inclinometers, microphones, cameras or other image sensors, proximity sensors, motion sensors, gyroscopes, accelerometers, thermal sensors, humidity sensors, barometers, fingerprint scanners, iris scanners, pedometers, heart rate monitors, pulse oximeters, touch screens, near-field communication (“NFC”) sensors, radio-frequency identification (“RFID”) sensors, air gesture sensors, 3D (“3 Dimensional”) air gesture sensors, 3D scanners, Global Positioning System (“GPS”) units, clocks, EEG, ECG/EKG, EMG and others.

Layer 3: Data interpretation: Once sensor data has been captured, it needs to be processed and interpreted for usability. Using eye gaze tracking as an example, the duration and location of a user's gaze across a presented image stimulus must be converted front a machine code data sequence into (in one possible implementation) an XY-coordinate plane that represents the screen and the image upon it, as well as the length of time the user spent gazing at each XY-coordinate or sector of coordinates.

Layer 4: Application software and portable data: To establish a neuropsychological profile for a smart device user based on their behavior patterns as they interact with their smart device, interpreted data from sensor arrays (in one implementation) must be collapsed into discrete classifications with relative scores; much as neuropsychological tests do traditionally. For example, if the image presented in our theoretical case is the image of a red-haired model. Over time and over a series of images of different models, a software solution using the methods of this innovation will be able to determine patterns of perceptual response the user exhibits when presented with this stimulus.

If, for example, the user spends 0.5 fewer seconds looking at the red-haired models when compared to images of blonde-haired models, it may be evidence of an implicit bias either away from red-haired models or towards blonde-hair models. If these datasets were combined over time, a scale between −1.00 and +1.00 can represent this implicit bias (as an example implementation). If a score of −1.00 represents a bias of >2.0 seconds towards looking at blonde-haired models over red-haired models and +1.00 represents a bias of >2.0 seconds towards looking at red-haired models over blonde-haired models, this user would be rated at −0.25.

This data point can then, for example, be shared with advertisers and content creators who could then make informed decisions regarding which images and representations they show to this specific user. If the goal of the advertiser or content creator is to expose this user to more images of red-haired models, they are able to do this and assess the user's response. If, alternatively, the goal of the advertiser or content creator is to show the user more images of what they are already naturally attracted to, they are able to do this as well; and accordingly assess the user's response.

Additionally, advertisers and content creators can target specific bands of users when this data is aggregated. For example, consider focusing ads on people with an implicit bias towards blonde-haired models at the level of +0.75 to +1.00 for a specific advertisement. Population level analysis is now also possible and can be easily conducted. Consider, for example, of all applicable users in Las Vegas, Nev. if there is skew of +0.10 bias towards the preference of red-haired models.

Each sensor on board a smart device is able to be repurposed to derive specific user brain functioning behavior. The system and general methods for accomplishing this are considered within the scope of the present invention.

In various embodiments of the innovation, inclinometer/gyroscopes may be utilized for determining the angle at which a device is being held. Which can determine a user's predicted posture (sitting down, laying down, neck crooked downwards, etc.) and strength/stability of a user's grip/arm control. They can be used to determine the duration and magnitude of a user's motion over time. They can be used to predict whether a user has oriented a device towards or away from their vision.

In various embodiments of the innovation, microphones may be utilized for collecting background noise level data, voice capture, sounds emitted from the body (such as coughing, sneezing, sniffling, etc.), etc.

In various embodiments of the innovation, a camera/image sensor/iris scanner may be utilized for eye tracking/gaze analysis, saccade analysis, fatigue monitoring, attentional control, facial expression, information tracking (to include focus, attention, emotional expression, etc.), ambient light level analysis, etc.

In various embodiments of the innovation, proximity sensors may detect how close or far the user holds the device from their face.

In various embodiments of the innovation, motion sensors may be utilized to determine how the user moves the device while engaged in certain activities. They can also detect if a user is staring at a screen for too long without a break. They may also be used to collect and compare with data from other sensors.

In various embodiments of the innovation, a heart rate monitor may be utilized to detect changes in a user's heart rate for various purposes (e.g. as a reaction to specific stimuli, when compared with GPS data to determine stressful or relaxing physical locations, etc.).

In various embodiments of the innovation, a touch screen may be utilized for wide ranging analysis of tactile input (e.g. pattern detection).

In various embodiments of the innovation, air gesture or 3D scanners can detect markers of clinical symptomatology.

In various embodiments of the innovation GPS sensors can correlate location data with other data derived from various sensors.

An example implementation can detect significant decrements in cognitive functioning in certain environments before traditional cognitive test batteries and before the impairment manifest as impaired performance. Monitoring psychomotor activity, oculomotor activity, and P300 response latency and signal amplitude can be indictive of fatigue.

Psychomotor activity is monitored because as fatigue increases motor movement precision decreases. Oculomotor activity is monitored because as fatigue increases eye saccadic velocity decreases and eye blinks increase in both frequency and duration. P300 response is monitored because fatigue increases the latency of this neurophysiological response and its amplitude decreases.

Use of a sys-app or sub-app on a device allows data collection without the test subject's conscious control of their responses. There are two simple methods to access a user's degree of psychomotor precision while using a device. For each method baseline data, averages and deviations are considered important.

Misclick analysis: Misclicks are defined here as ‘mousedown’ and ‘mouseup’ clicks that land outside of a defined area on the graphical user interface (GUI). When a user accidentally or unintentionally clicks outside the containment border of a program's interactive element, this is defined as a misclick.

For example, if a user clicks a few pixels to the right of the ‘OK’ button on a program, this is considered a misclick. The ratio of successful clicks to misclicks can provide insight into a user's level of psychomotor control and precision. As fatigue increases, the ratio of successful clicks to misclicks should decrease.

Cursor tracking analysis: Cursor tracking is defined here as the degree and magnitude of a mouse cursor's deflection from the center of a program's interactive element on the GUI immediately preceding a mousedown mouse click. This value can provide insight into the accuracy of a user's fine psychomotor muscle control. As fatigue increases, mouse cursor deflection from the center of a program's interactive elements on the GUI should also increase.

Oculomotor Activity: The eyes provide a wealth of data regarding a person's neuropsychological status. Not only can the eyes indicate a user's intention in gaze tracking analysis, but they can also indicate fatigue by way of aberrant oculomotor activity. There are five methods to assess a user's neuropsychological status and functioning while using a device. For each method baselines, averages and deviations are considered important.

Eye gaze tracking: Eye gaze tracking is defined here as the method of determining where a user is looking at a screen. As fatigue increases, total surface “gazed” upon by the users eyes per unit time is likely to decrease.

Eye saccade tracking: Eye saccade tracking is defined here as the method of determining the speed of the eye's natural saccade. As fatigue increases, the speed of the eye's natural saccade decreases.

Ocular surface area exposure: Ocular surface area exposure is defined here as the method of determining how open or closed a user's eyelids are. As fatigue increases, the total exposed surface area of the eye decreases (due to eyelid closure).

Eye blink count: Eye blink count is defined here as the method of determining how many times a user blinks per unit time. As fatigue increases, eye blink frequency and duration also increase.

Screen attentiveness: Screen attentiveness is defined here as the method of determining when a user looks away from the screen. This measure may not provide evidence of fatigue, but it may serve as a measure of distractibility or irritation.

Electroencephalogram: The P300 response is a classically reliable indicator of attention; especially within what is referred to as the “oddball” paradigm. As an individual's attentiveness decreases, response latencies to the “oddball” stimulus increase while the amplitude of the positive deflecting neural waveform decreases.

### BRIEF DESCRIPTION OF THE DRAWINGS

FIG. 1 illustrates the input, process, and output structure of the innovation in accordance with an exemplary embodiment of the invention.

FIG. 2 illustrates detection through various sensors of user interaction with a device in accordance with an exemplary embodiment of the invention.

FIG. 3 shows use and flow of data for visual heat mapping in accordance with an exemplary embodiment of the invention.

FIG. 4 shows handling of stimulus responses from hardware to software in accordance with various exemplary embodiments of the invention.

### DETAILED DESCRIPTION OF THE DRAWINGS

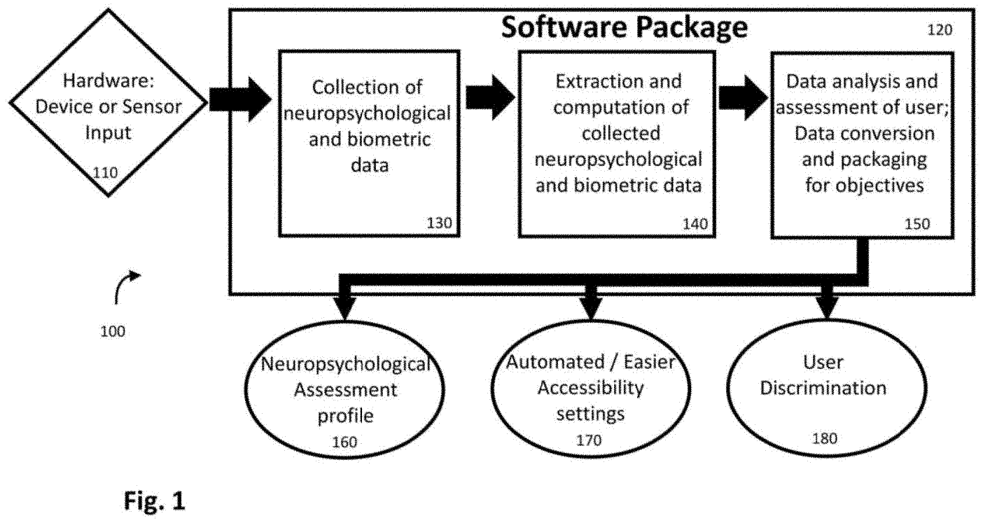

FIG. 1 illustrates the input, process, and output structure of the innovation in accordance with an exemplary embodiment of the invention. The diagram 100 shows the interactions of a software package 120 which may be a sys-app, or a sub-app as described above. The software package 120 receives hardware device or sensor input 110. The data received is collected to form a collection of neuropsychological and biometric data 130 associated with a stimulus or initiating event.

The collection of neuropsychological and biometric data 130 is extracted and analyzed through various computations 140 to create a useable dataset. The useable dataset contains: data analysis and assessment of the user; packaged with data conversions and/or raw data according to the objectives of the data's use 150. The packaged data, which may include one or more of raw data streams, computations of data, and/or user assessments and analysis of the neuropsychological and biometric data 150, is transmitted or otherwise utilized locally or remotely. The data package is formatted for a neuropsychological assessment profile objective 160, configuration for automated/easier accessibility settings 170, or user discrimination for security purposes 180.

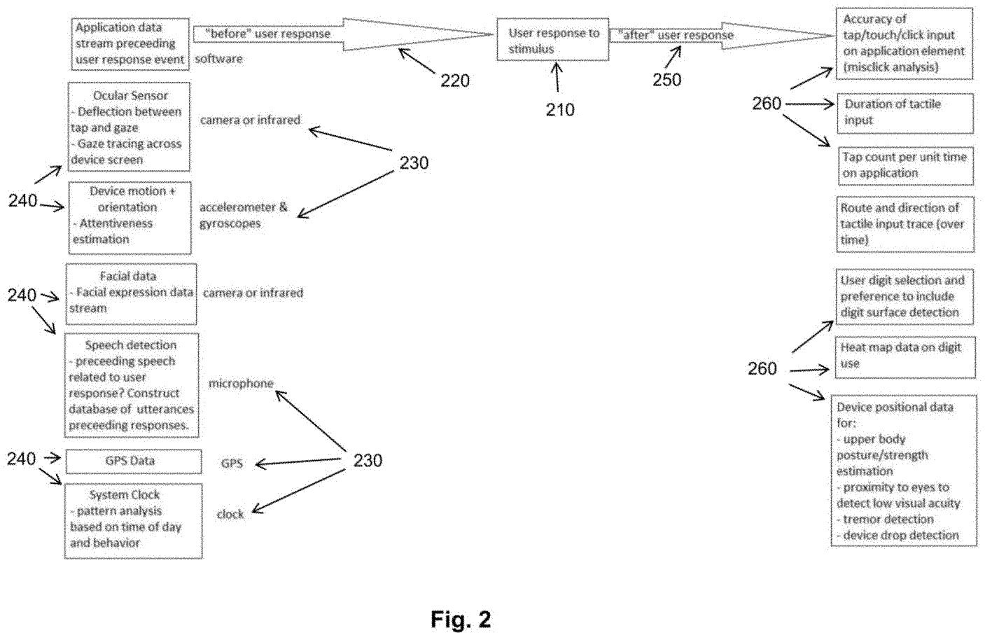

FIG. 2 illustrates detection through various sensors of user interaction with a device in accordance with an exemplary embodiment of the invention. A user response to stimulus 210, i.e. a stimulus response may be determined by data collected from sensor devices 230 after the stimulus 250, or it may be determined by data collected from sensor devices 230 before the stimulus 220.

This means some stimulus 210 or initiating events would utilize data 240 from sensors 230 collected before 220 the event 210. In this sort of event, it may be more relevant to determine what activity caused the event.

However, some stimulus 210 or initiating events would utilize data 260 from sensors 230 collected after 250 the event 210. In this sort of event, it may be more relevant to determine what activity the triggering event may cause as a result.

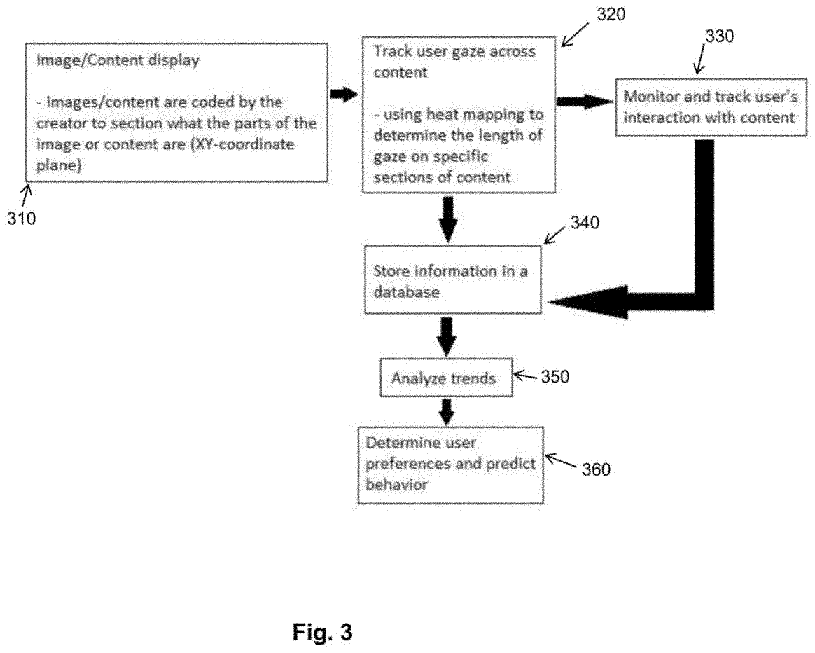

FIG. 3 shows use and flow of data for visual heat mapping in accordance with an exemplary embodiment of the invention. Images content are coded by the creator to section what the parts of the image or contents are (XY-coordinate plane) 310. Identifying the location of specific image contents and delineating the boundaries between different content allows the particular coordinates of a user's gaze to be coordinated with particular content the user is viewing. By tracking eye gaze, and recording time spend on various contents 320, various inferences may be made.

As a user's gaze is tracked and mapped to specific content sections 320, the data can be logged to a data collection 340. This data can also be analyzed in relation to the specific content 330 to make different determinations about the user. It may also stimulate particular user actions, which may also be useful data, and should be stored 340. Data from multiple images/different content can yield further results such as trending 350, which may be useful in determining user preferences and/or predicting behavior 360.

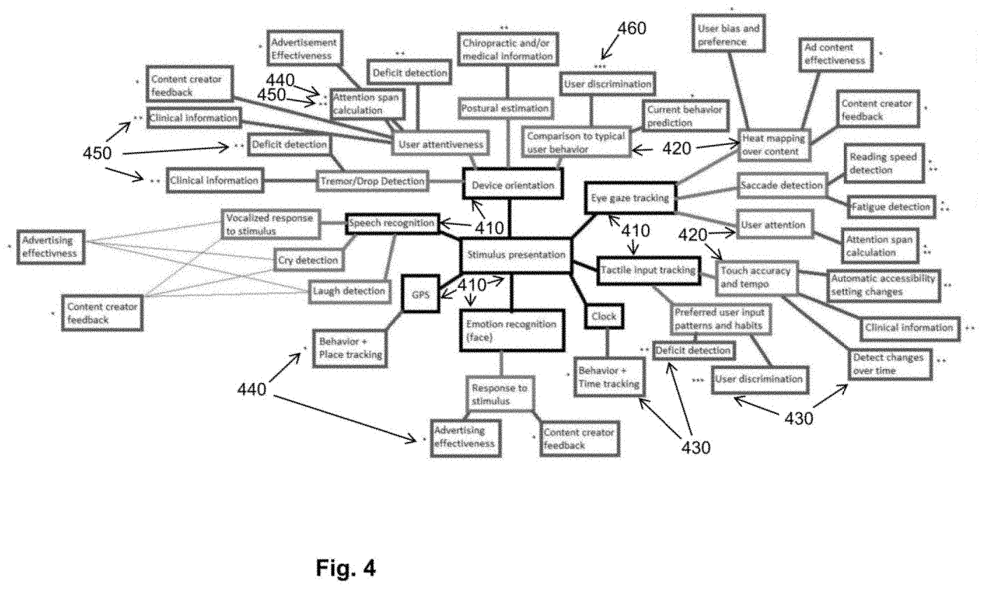

FIG. 4 shows handling of stimulus responses from hardware to software in accordance with various exemplary embodiments of the invention. Hardware level sensors 410 generate data that collected and interpreted. The interpretations, with or without the raw data, is packaged into an interpretation 420. The interpretation 420 is then utilized and/or further analyzed 430 for purposes related to: 1) analytics for advertisers or content creators 440; 2) clinical/medical/neuropsychological uses 450; and/or 3) device security and configuration 460.

The flow diagrams in accordance with exemplary embodiments of the present invention are provided as examples and should not be construed to limit other embodiments within the scope of the invention. For instance, the blocks should not be construed as steps that must proceed in a particular order. Additional blocks/steps may be added, some blocks/steps removed, or the order of the blocks/steps altered and still be within the scope of the invention. Further, blocks within different figures can be added to or exchanged with other blocks in other figures. Further yet, specific numerical data values (such as specific quantities, numbers, categories, etc.) or other specific information should be interpreted as illustrative for discussing exemplary embodiments. Such specific information is not provided to limit the invention.

In the various embodiments in accordance with the present invention, embodiments are implemented as a method, system, and/or apparatus. As one example, exemplary embodiments are implemented as one or more computer software programs to implement the methods described herein. The software is implemented as one or more modules (also referred to as code subroutines, or “objects” in object-oriented programming). The location of the software will differ for the various alternative embodiments. The software programming code, for example, is accessed by a processor or processors of the computer or server from long-term storage media of some type, such as a CD-ROM drive or hard drive. The software programming code is embodied or stored on any of a variety of known media for use with a data processing system or in any memory device such as semiconductor, magnetic and optical devices, including a disk, hard drive, CD-ROM, ROM, etc. The code is distributed on such media or is distributed to users from the memory or storage of one computer system over a network of some type to other computer systems for use by users of such other systems. Alternatively, the programming code is embodied in the memory (such as memory of the handheld portable electronic device) and accessed by the processor using the bus. The techniques and methods for embodying Software programming code in memory, on physical media, and/or distributing software code via networks are well known and will not be further discussed herein.

The above discussion is meant to be illustrative of the principles and various embodiments of the present invention. Numerous variations and modifications will become apparent to those skilled in the art once the above disclosure is fully appreciated. It is intended that the following claims be interpreted to embrace all such variations and modifications.

What is claimed is:

1. A system for the active electronic collection of neuropsychologically and biometrically relevant data from sensors of a computing device, wherein data collected from sensors is correlated to a triggering event and analyzed to determine specific brain states and functions associated to the triggering event.

2. The system described in claim 1 wherein the data is packaged for storage and/or transmission to a remote computing device.

3. A system for the passive electronic collection of neuropsychologically and biometrically relevant data from sensors of a computing device, wherein data collected from sensors is correlated to a triggering event and analyzed to determine specific brain states and functions associated to the triggering event.

4. The system described in claim 3 wherein the data is packaged for storage and/or transmission to a remote computing device.

5. A method for administering neuropsychological tests on a computing device comprising:
configuring the device to administer a neuropsychological test; and
configuring the device to collect and record data related to administering the test;
wherein the data related to administering the test comprises one or more of:
correct test responses;
incorrect test responses;
time between presentation of and response to one or more distinct components of the test.

6. The method described in claim 5 wherein data related to administering the test further comprises:
relevant and concurrent biometric data.

7. A method to create an objective neuropsychological and/or biometric profile of an individual comprising:
configuring a computing device to administer a neuropsychological test;
configuring the device to collect and record data related to administering the test;
wherein the data related to administering the test comprises one or more of:
correct test responses;
incorrect test responses;
time between presentation of and response to one or more distinct components of the test;
optionally monitoring and collecting relevant and concurrent biometric data during test administration; and
analyzing the data to develop a profile of the individual.

8. A method to create an objective neuropsychological and/or biometric profile of an individual comprising:
configuring a computing device to collect and record passive sensor data during an individual's usage of the device;
identifying one or more stimulus or initiating events occurring during the individual's usage; and
analyzing collected data related to identified stimulus or initiating events to develop a profile of the individual.

9. A method to create an objective neuropsychological and/or biometric profile of an individual comprising:
configuring a computing device to collect and record active sensor data during an individual's usage of the device;
identifying one or more stimulus or initiating events occurring during the individual's usage; and
analyzing collected data related to identified stimulus or initiating events to develop a profile of the individual.

10. A method to automatically configure a computing device comprising:
configuring the computing device to collect and record sensor data during an individual's usage of the device;
identifying one or more stimulus or initiating events occurring during the individual's usage;
analyzing collected data related to identified stimulus or initiating events to develop a profile of the individual;
identifying and performing configuration changes to the device in response to the developed profile of the individual.

11. A method to differentiate native users from other users of a computing device comprising:
configuring the computing device to collect and record sensor data during an individual's usage of the device;
identifying one or more stimulus or initiating events occurring during the individual's usage;
analyzing collected data related to identified stimulus or initiating events to develop a profile of the individual;
storing the profile of the individual;
collecting and recording sensor data during continued usage of the device;
identifying one or more stimulus or initiating events occurring during the continued usage;
analyzing collected data related to identified stimulus or initiating events during the continued usage to develop a second profile of the current individual;
comparing the first profile to the second profile to determine differences indicating the profiles are of different individuals.

12. A unifying protocol for the transmission and receipt of data collected from onboard sensor arrays and software data, wherein the data collected is packaged uniformly for storage and/or transmission.

13. The unifying protocol as described in claim 12 wherein received packages of sensor data are unpackaged to form a collection of data that is processed.
